(19) United States
(12) Patent Application Publication
(10) Pub. No.: US 2018/0272327 A1
(43) Pub. Date: Sep. 27, 2018

(54) IONIC LIQUID CATALYST TREATING SYSTEM

(71) Applicant: UOP LLC, Des Plaines, IL (US)

(72) Inventors: Matthew Lippmann, Oak Park, IL (US); David S. Lafyatis, Schaumburg, IL (US); Douglas A. Nafis, Mount Prospect, IL (US); Kurt Detrick, Glen Ellyn, IL (US)

(21) Appl. No.: 15/994,962

(22) Filed: May 31, 2018

Related U.S. Application Data
(63) Continuation of application No. PCT/US2016/063079, filed on Nov. 21, 2016.
(60) Provisional application No. 62/268,865, filed on Dec. 17, 2015.

(51) Int. Cl. B01J 31/40; B01J 31/02; B01J 38/64; C07C 7/12; C07C 7/13; C07C 7/00; C07C 7/10; C10G 25/02; C10G 25/05; C10G 53/08; C10G 31/00; B01D 17/04; B01D 17/02; B01D 17/06

(52) U.S. Cl. CPC B01J 31/40; C10G 2300/4081; B01J 31/0298; B01J 31/0288; B01J 38/64; C07C 7/12; C07C 7/13; C07C 7/005; C07C 7/10; C10G 25/02; C10G 25/05; C10G 53/08; C10G 31/00; B01D 17/045; B01D 17/0202; B01D 17/0208; B01D 17/06; C10G 2300/706; B01J 31/0282

(57) ABSTRACT

A process removing ionic liquid from a process stream is described. The process stream is introduced into a coalescer to form an ionic liquid stream and a first treated process stream which has less ionic liquid than the process stream. The first treated process stream is introduced into a separator to form a second treated process stream. The second treated process stream has less ionic liquid than the first treated process stream. The separator is selected from a filtration zone comprising sand or carbon, an adsorption zone, a scrubbing zone, an electrostatic separation zone, or combinations thereof.

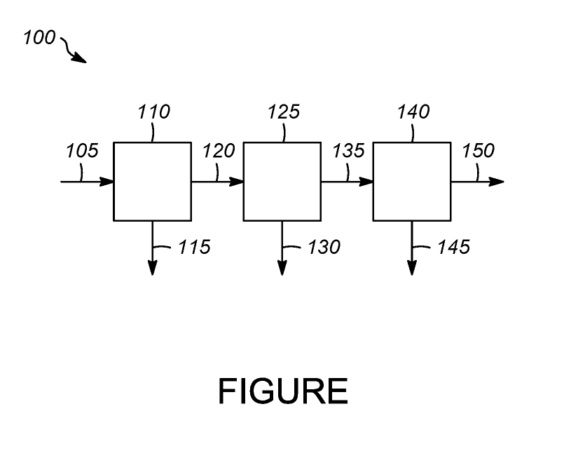

### CROSS-REFERENCE TO RELATED APPLICATIONS

This application is a Continuation of copending International Application No. PCT/US2016/063079 filed Nov. 21, 2016, which application claims priority from U.S. Provisional Application No. 62/268,865 filed Dec. 17, 2015, now expired, the contents of which cited applications are hereby incorporated by reference in their entirety.

### BACKGROUND OF THE INVENTION

Various hydrocarbon conversion processes can utilize ionic liquid catalysts. Alkylation is typically used to combine light olefins, for example mixtures of alkenes such as propylene and butylene, with isobutane to produce a relatively high-octane branched-chain paraffinic hydrocarbon fuel, including isoheptane and isooctane. Similarly, an alkylation reaction can be performed using an aromatic compound such as benzene in place of the isobutane. When using benzene, the product resulting from the alkylation reaction is an alkylbenzene (e.g. ethylbenzene, cumene, dodecylbenzene, etc.).

Processes for the oligomerization of light olefins (e.g. ethylene, propylene, and butylene) to produce higher carbon number olefin products (e.g. C₆₊ olefins) are well known. Oligomerization processes have been employed to produce high quality motor fuel components as well as petrochemicals from ethylene, propylene, and butylene. These oligomerization processes are also referred to as catalytic condensation and polymerization, with the resulting motor fuel often referred to as polymer gasoline.

The disproportionation of paraffins (e.g., isopentane (iC₅)) involves reacting two moles of hydrocarbon to form one mole each of two different products, one having a carbon count greater than the starting material and the other having a carbon count less than the starting material. The total number of moles in the system remains the same throughout the process, but the products have different carbon counts from the reactants.

Isomerization of linear paraffins to their branched isomers increases their octane number and thus their value to a refiner. Isomerization processes involve reacting one mole of a hydrocarbon (e.g., normal pentane) to form one mole of an isomer of that specific hydrocarbon (e.g., isopentane). The total number of moles remains the same throughout this process, and the product has the same number of carbons as the reactant.

Acidic ionic liquids can be used as an alternative to the commonly used strong acid catalysts in hydrocarbon conversion processes. Ionic liquids are catalysts that can be used in a variety of catalytic reactions, including the alkylation of paraffins with olefins. Ionic liquids are salts comprised of cations and anions which typically melt below about 100° C.

Ionic liquids are essentially salts in a liquid state, and are described in U.S. Pat. Nos. 4,764,440, 5,104,840, and 5,824,832. The properties vary extensively for different ionic liquids, and the use of ionic liquids depends on the properties of a given ionic liquid. Depending on the organic cation of the ionic liquid and the anion, the ionic liquid can have very different properties.

Ionic liquids provide advantages over other catalysts, including being non-volatile.

Ionic liquids have also been used in separation processes, such as the removal of various contaminants from hydrocarbons as described in U.S. Pat. Nos. 7,749,377, 8,574,426, 8,574,427, 8,580,107, 8,608,943, 8,608,949, 8,608,950, 8,608,951, 8,709,236, for example, and the removal of contaminants from oxidation products as described in U.S. Pat. Nos. 8,754,254, 9,000,214, for example.

However, the use of ionic liquids presents unique and novel waste handling challenges due to the nature of the chemicals and compounds specific to the normal operation of the unit. Many of these substances, including but not limited to the ionic liquid itself, are not suitable to be released, drained, or otherwise discharged into standard refinery relief systems, waste handling systems, or other similar systems intended and designed to manage waste or unit non-product streams.

For example, U.S. Pat. No. 8067,656 describes a process for separating ionic liquid from hydrocarbons using a coalescer. The process comprises: (a) feeding a mixture comprising hydrocarbons and ionic liquid to a coalescer, the hydrocarbons having ionic liquid droplets dispersed therein, and the coalescer comprising a coalescer material; (b) adhering at least a portion of the ionic liquid droplets to the coalescer material to provide captured droplets; (c) coalescing captured droplets into coalesced droplets; and (d) allowing the coalesced droplets to fall from the coalescer material to separate the ionic liquid from the hydrocarbons and provide a hydrocarbon effluent, wherein the coalescer material has a stronger affinity for the ionic liquid than the hydrocarbons. The droplets fall to the bottom of the coalescer and form an ionic liquid layer which can then be removed from the coalescer. Multiple stages of coalescer material can be used in series, in parallel, or both. The stages can have different size openings in the coalescer material. The hydrocarbon effluent is said to comprise 40 ppm or less of ionic liquid, or 20 ppm or less of ionic liquid, or 10 ppm or less of ionic liquid.

However, it has been discovered that in some embodiments those levels of ionic liquid are still too high. For example, it has been discovered that levels of 10 to 40 ppm of ionic liquid may still be toxic for microbes in wastewater treatment facilities. Product streams from various processes may also need to have ionic liquid removed before being sent for storage or use.

Therefore, there remains a need for a system for treating streams containing ionic liquid catalysts.

### SUMMARY OF THE INVENTION

One aspect of the present invention is a process removing ionic liquid from a process stream. In one embodiment, the process involves introducing the process stream into a coalescer to form an ionic liquid stream and a first treated process stream which has a level of ionic liquid less than the level of ionic liquid in the process stream. The first treated process stream is introduced into a separator to form a second treated process stream, the second treated process stream having a level of ionic liquid less than the level of ionic liquid in the first treated process stream. The separator is selected from a filtration zone comprising sand or carbon, an adsorption zone, a scrubbing zone, an electrostatic separation zone, or combinations thereof.

### BRIEF DESCRIPTION OF THE DRAWING

The FIGURE illustrates one embodiment of a process for removing ionic liquid from a process stream.

### DETAILED DESCRIPTION OF THE INVENTION

The FIGURE illustrates one embodiment of a process 100 for removing ionic liquid from a process stream. The process stream can be any type of process stream which contains ionic liquid, including, but not limited to, organic streams, and inorganic streams. For convenience, the FIGURE will be described with respect to a hydrocarbon conversion process that has been facilitated by an ionic liquid

Most hydrocarbon conversion reactions or contaminant removal using ionic liquids are biphasic and take place at the interface in the liquid state due to the low solubility of hydrocarbons in ionic liquids.

Although the reaction or removal will proceed simply by contacting the hydrocarbon feed and the ionic liquid catalyst, the reaction or removal rate by contacting alone may be too slow to be commercially viable. Consequently, the hydrocarbon feed and the ionic liquid are often mixed to provide better contact. The mixing produces a dispersion of ionic liquid droplets in the hydrocarbon. The dispersed ionic liquid droplets need to be removed from the hydrocarbon stream.

The hydrocarbon feed stream 105 containing the dispersed ionic liquid droplets is sent to an optional gravity settler 110. Separation occurs as a result of the density difference between the ionic liquid and hydrocarbon. The lighter hydrocarbon phase is located above the heavier ionic liquid phase. The ionic liquid phase can be removed from the gravity settler as a first ionic liquid stream 115.

The lighter hydrocarbon phase is removed from the gravity settler 110 as settler effluent stream 120, which has a lower level of ionic liquid than the incoming hydrocarbon feed stream 105.

The settler effluent stream 120 is sent to a coalescer 125. The coalescer 125 is a device having a suitable material to facilitate separation of immiscible liquids. The coalescer 125 typically contains at least one of: one or more metal wires, one or more vanes, metal mesh or packing, one or more glass or polymer fibers, glass beads, sand, anthracite coal, and ceramic membrane. These components may be constructed of or coated with materials that exhibit hydrophobic-oleophilic characteristics. The coalescer can be static. Alternatively, it can be an active coalescer, such as described in U.S. application Ser. No. 14/700,919, entitled Active Coalescer to Remove Fine Particles, filed Apr. 30, 2015, which is incorporated herein by reference.

Droplets of ionic liquid contact the coalescer material in the coalescer 125 and form larger drops on the coalescer material. These droplets then fall to the bottom of the coalescer 125 if the density of the droplet is more than the density of the hydrocarbon and form a layer of ionic liquid in the bottom of the coalescer 125. The ionic liquid can be removed as a second ionic liquid stream 130.

The coalescer effluent 135 from the coalescer 125 still contains dispersed ionic liquid droplets. The level of ionic liquid in the coalescer effluent 135 is less than the level in the settler effluent 120. However, it was discovered that this level is still too high to permit the coalescer effluent 135 to be released to a standard wastewater treatment facility. The ionic liquid in the coalescer effluent 135 may also be too high for use in various products. For example, internal combustion engines may not tolerate ppm levels of ionic liquid impurities in gasoline or diesel fuels. Chemicals for use eventually as polymers may not tolerate ppm quantities of ionic liquid, which could have deleterious impacts on the polymerization processes. Fine chemical or pharmaceutical applications may not tolerate ppm quantities of ionic liquid due to regulatory restrictions.

Consequently, the coalescer effluent 135 must be further treated in a separator 140. First separator 140 can be one or more of a filtration zone comprising sand or carbon, an adsorption zone, a scrubbing zone, an electrostatic separation zone, or combination thereof. The separator can include one or more of one type of separator followed by one or more of a different type of separator. For example, there could be two filtration zones, followed by three adsorption zones, followed by a scrubbing zone.

The filtration zone comprises a vessel which contains a fixed bed of sand or carbon particles in the top section of the vessel and a separation zone in the bottom section of the vessel. The coalescer effluent stream 135 enters the top of the vessel, and as the liquid passes through the fixed bed of sand or carbon particles, some of the small ionic liquid droplets coalesce into larger droplets. These larger droplets then settle to the bottom of the separation zone to form a layer of ionic liquid in the bottom of the vessel which can be extracted as a third ionic liquid stream 145. The hydrocarbon is removed from the side of the separation zone near the bottom of the vessel as stream 150 or it may flow to additional filtration, adsorption, or scrubbing zones. The sand or carbon particles are sized appropriately to coalesce some of the remaining small droplets of ionic liquid that are present in the coalescer effluent stream 135.

The adsorption zone comprises an adsorbent bed containing an adsorbent. In some embodiments, the adsorbent comprises at least one of oxides and oxide materials such as silica, silica gel, glass, glass beads, sand, and alumina could be used as adsorbents in granular, fiber, pellet, or other form. Salts, such as MgSO₄ and CaSO₄, that are traditionally used as drying agents could be used as adsorbent material. Other salts could adsorb ionic liquid as well due to charge-dipole and dipole-dipole interactions. Ion exchange resins such as sulfonic acid resins would also be a possible adsorbent, as could fiber materials such as heteroatom containing polymers like Nylon-6 and other fibers such as wool. It is also believed that activated carbon and clays could be utilized as adsorbents. Zeolites could also be used as an adsorbent.

When the adsorbent is spent, a desorbent can be introduced to desorb the ionic liquid from the adsorbent. In another embodiment, the adsorbent bed could be heated to remove the desorbent. Alternatively, the adsorbent can be replaced, and the spent adsorbent can be disposed of.

The adsorbent zone may comprise multiple vessels with beds in a swing configuration or a lead lag configuration. Alternatively, the adsorbent zone could be a single vessel operated in alternating modes of adsorption and desorption. The beds may be fluidized or fixed beds.

In some embodiments, the scrubbing zone comprises at least one of water, and caustic. The scrubbing zone can comprises a vessel containing one or more trays, and/or distributor plates.

One example of a suitable electrostatic separation zone is described in U.S. application Ser. No. 62/081702, entitled Ionic Liquid Recovery From a Hydrocarbon Stream Using Electrostatic Force, filed Nov. 19, 2014, now abandoned, which is incorporated herein by reference. The hydrocarbon stream with the dispersed ionic liquid droplets is fed to the electrostatic separator. The electrostatic separator contains electrodes which establish an electric field, and the hydrocarbon stream flows into the electric field. The electric field can be an alternating current (AC) field which induces polarization on the ionic liquid droplets causing them to increase their collision frequency and coalesce. There is also additional electrostatic force between the polarized droplets and the electrodes. Larger ionic liquid droplets fall to the bottom of the separator and are collected. The electric field can also be a direct current (DC) field which causes electrophoretic motion of the ionic liquid droplets, also causing increased collision frequency and therefore coalescence. Pulsed AC or DC fields may also be utilized.

In some embodiments, the ionic liquid removed from the coalescer effluent 135 in the separator 140 can be removed from the separator 140 as a third ionic liquid stream 145. In other embodiments, such as with an adsorbent, there may not be an ionic liquid stream.

The separator effluent 150 from the separator may have less than 40 ppmw ionic liquid, or less than 20 ppmw, or less than 10 ppmw, or less than 5 ppmw, or less than 3 ppmw, or less than 1 ppmw.

In some embodiments, the separator effluent 150 from the separator 140 can be sent to a general waste treatment facility as needed (not shown) if the level of ionic liquid is sufficiently low. In other embodiments, the separator effluent can be sent to a product storage facility or used in additional processes, for example.

One or more of the first, second, and third ionic liquid streams 115, 130, 145 can be recovered and recycled to a process zone (not shown) All or a portion of the ionic liquid in one or more of these streams can be regenerated and/or reactivated, as needed.

A variety of methods for regenerating ionic liquids have been developed. For example, U.S. Pat. No. 7,651,970; U.S. Pat. No. 7,825,055; U.S. Pat. No. 7,956,002; U.S. Pat. No. 7,732,363, each of which is incorporated herein by reference, describe contacting ionic liquid containing the conjunct polymer with a reducing metal (e.g., Al), an inert hydrocarbon (e.g., hexane), and hydrogen and heating to about 100° C. to transfer the conjunct polymer to the hydrocarbon phase, allowing for the conjunct polymer to be removed from the ionic liquid phase. Another method involves contacting ionic liquid containing conjunct polymer with a reducing metal (e.g., Al) in the presence of an inert hydrocarbon (e.g. hexane) and heating to about 100° C. to transfer the conjunct polymer to the hydrocarbon phase, allowing for the conjunct polymer to be removed from the ionic liquid phase. See e.g., U.S. Pat. No. 7,674,739 B2; which is incorporated herein by reference. Still another method of regenerating the ionic liquid involves contacting the ionic liquid containing the conjunct polymer with a reducing metal (e.g., Al), HCl, and an inert hydrocarbon (e.g. hexane), and heating to about 100° C. to transfer the conjunct polymer to the hydrocarbon phase. See e.g., U.S. Pat. No. 7,727,925, which is incorporated herein by reference. The ionic liquid can be regenerated by adding a homogeneous metal hydrogenation catalyst (e.g., (PPh₃)₃RhCl) to ionic liquid containing conjunct polymer and an inert hydrocarbon (e.g. hexane), and introducing hydrogen. The conjunct polymer is reduced and transferred to the hydrocarbon layer. See e.g., U.S. Pat. No. 7,678,727, which is incorporated herein by reference. Another method for regenerating the ionic liquid involves adding HCl, isobutane, and an inert hydrocarbon to the ionic liquid containing the conjunct polymer and heating to about 100° C. The conjunct polymer reacts to form an uncharged complex, which transfers to the hydrocarbon phase. See e.g., U.S. Pat. No. 7,674,740, which is incorporated herein by reference. The ionic liquid could also be regenerated by adding a supported metal hydrogenation catalyst (e.g. Pd/C) to the ionic liquid containing the conjunct polymer and an inert hydrocarbon (e.g. hexane). Hydrogen is introduced and the conjunct polymer is reduced and transferred to the hydrocarbon layer. See e.g., U.S Pat. No. 7,691,771, which is incorporated herein by reference. Still another method involves adding a suitable substrate (e.g. pyridine) to the ionic liquid containing the conjunct polymer. After a period of time, an inert hydrocarbon is added to wash away the liberated conjunct polymer. The ionic liquid precursor [butylpyridinium][Cl] is added to the ionic liquid (e.g. [butylpyridinium][Al₂Cl₇]) containing the conjunct polymer followed by an inert hydrocarbon. After mixing, the hydrocarbon layer is separated, resulting in a regenerated ionic liquid. See, e.g., U.S, Pat. No. 7,737,067, which is incorporated herein by reference. Another method involves adding ionic liquid containing conjunct polymer to a suitable substrate (e.g. pyridine) and an electrochemical cell containing two aluminum electrodes and an inert hydrocarbon. A voltage is applied, and the current measured to determine the extent of reduction. After a given time, the inert hydrocarbon is separated, resulting in a regenerated ionic liquid. See, e.g., U.S. Pat. No. 8,524,623, which is incorporated herein by reference. Ionic liquids can also be regenerated by contacting with silane compounds (U.S. Pat. No. 9,120,092), borane compounds (U.S. Publication No.2015/0314281), Brønsted acids, (U.S. Pat. No. 9,079,176), or C₁ to C₁₀ Paraffins (U.S. Pat. No. 9,079,175), each of which is incorporated herein by reference.

### EXAMPLES

### Example 1

10.1 grams of a 1-butyl-3-methylimidazolium hexafluorophosphate ionic liquid (BMIM) was added to 720 grams of deionized (DI) water, giving a pH of about 3. This solution was then neutralized with approximately 4.9 grams of NaOH, forming a solid precipitate of mostly aluminum hydroxide, and bringing the solution pH to approximately 6. After filtration to remove the precipitate, this solution, which contains approximately 0.014 mg of ionic liquid/mg solution, was tested for toxicity. The half maximal effective concentration (EC-50) for this solution was then determined for the luminescence of Aliivibrio fischeri bacteria to assess the toxicity of the solution. It was determined that the EC-50 was reached at a dilution of 5.7 mg solution/Liter water. Thus, the EC-50 for this ionic liquid was approximately 80 ppB, far below the level believed to be achievable through merely combining gravity settling with a coalescer.

### Example 2

10.1 grams of a tri-butyl pentyl phosphonium ionic liquid (TBPP) was added to 770 grams of deionized (DI) water, giving a pH of about 3. This solution was then neutralized with approximately 4.3 grams of NaOH, forming a solid precipitate of mostly aluminum hydroxide, and bringing the solution pH to approximately 6. After filtration to remove the precipitate, this solution, which contains approximately 0.013 mg of ionic liquid/mg solution, was tested for toxicity. The EC-50 for this solution was then determined for the luminescence of Aliivibrio fischeri bacteria to assess the toxicity of the solution. It was determined that the EC-50 was reached at a dilution of 1.2 mg solution/Liter water. Thus, the EC-50 for this ionic liquid was approximately 16 ppB, far below the level believed to be achievable through merely combining gravity settling with a coalescer.

By the term “about,” we mean within 10% of the value, or within 5%, or within 1%.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

### Specific Embodiments

While the following is described in conjunction with specific embodiments, it will be understood that this description is intended to illustrate and not limit the scope of the preceding description and the appended claims.

A first embodiment of the invention is a process for removing ionic liquid from a process stream comprising introducing the process stream into a coalescer to form an ionic liquid stream and a first treated process stream having a level of ionic liquid less than a level of ionic liquid in the process stream; and introducing the first treated process stream into a separator to form a second treated process stream, the second treated process stream having a level of ionic liquid less than the level of ionic liquid in the first treated process stream, the separator selected from a filtration zone comprising sand or carbon, an adsorption zone, a scrubbing zone, an electrostatic separation zone, or combinations thereof. An embodiment of the invention is one, any or all of prior embodiments in this paragraph up through the first embodiment in this paragraph wherein the level of ionic liquid in the second treated process stream is less than about 40 ppmw. An embodiment of the invention is one, any or all of prior embodiments in this paragraph up through the first embodiment in this paragraph wherein the level of ionic liquid in the second treated process stream is less than about 20 ppmw. An embodiment of the invention is one, any or all of prior embodiments in this paragraph up through the first embodiment in this paragraph wherein the level of ionic liquid in the second treated process stream is less than about 5 ppmw. An embodiment of the invention is one, any or all of prior embodiments in this paragraph up through the first embodiment in this paragraph wherein the separator is an adsorption zone, and wherein the adsorption zone contains an adsorbent comprising at least one of an oxide, a salt, an ion exchange resin, a polymer, a fiber material, activated carbon, clay, a molecular sieve, a zeolite, or combinations thereof. An embodiment of the invention is one, any or all of prior embodiments in this paragraph up through the first embodiment in this paragraph further comprising desorbing the ionic liquid from the adsorbent with a desorbent or by heating. An embodiment of the invention is one, any or all of prior embodiments in this paragraph up through the first embodiment in this paragraph wherein the separator is the scrubbing zone, and wherein the scrubbing zone contains at least one of water and caustic. An embodiment of the invention is one, any or all of prior embodiments in this paragraph up through the first embodiment in this paragraph wherein the scrubbing zone comprises a vessel containing a tray, a distributor plate, or combinations thereof. An embodiment of the invention is one, any or all of prior embodiments in this paragraph up through the first embodiment in this paragraph further comprising introducing a process feed stream into a gravity settler to form the process stream and a gravity settler ionic liquid stream before introducing the process stream into the coalescer. An embodiment of the invention is one, any or all of prior embodiments in this paragraph up through the first embodiment in this paragraph further comprising recovering at least one of the ionic liquid stream from the coalescer, and an ionic liquid stream from the separator. An embodiment of the invention is one, any or all of prior embodiments in this paragraph up through the first embodiment in this paragraph further comprising at least one of regenerating at least a portion of the recovered ionic liquid; and recycling at least a portion of the recovered ionic liquid to a process zone. An embodiment of the invention is one, any or all of prior embodiments in this paragraph up through the first embodiment in this paragraph further comprising at least one of passing the second treated process stream to a storage facility; passing the second treated process stream to a reaction zone as a feed stream; passing the second treated process stream to a waste treatment facility; and recovering the second treated process stream as a final product.

A second embodiment of the invention is a process for removing ionic liquid from a process stream comprising introducing a process feed stream into a gravity settler to form a process stream having a level of ionic liquid less than a level of ionic liquid in the process feed stream and an ionic liquid stream; introducing the process stream into a coalescer to form a second ionic liquid stream and a first treated process stream having a level of ionic liquid less than the level of ionic liquid in the process stream; introducing the first treated process stream into a separator to form a second treated process stream, the second treated process having a level of ionic liquid less than about 40 ppmw, the separator selected from a filtration zone comprising sand or carbon, an adsorption zone, a scrubbing zone, an electrostatic separation zone, or combinations thereof. An embodiment of the invention is one, any or all of prior embodiments in this paragraph up through the second embodiment in this paragraph wherein the level of ionic liquid in the second treated process stream is less than about 20 ppmw. An embodiment of the invention is one, any or all of prior embodiments in this paragraph up through the second embodiment in this paragraph wherein the level of ionic liquid in the second treated process stream is less than about 5 ppmw. An embodiment of the invention is one, any or all of prior embodiments in this paragraph up through the second embodiment in this paragraph wherein the separator is the adsorption zone, and wherein the adsorption zone contains an adsorbent comprising at least one of an oxide, a salt, an ion exchange resin, a polymer, a fiber material, activated carbon, clay, a molecular sieve, a zeolite, or combinations thereof. An embodiment of the invention is one, any or all of prior embodiments in this paragraph up through the second embodiment in this paragraph wherein the separator is the scrubbing zone, and wherein the scrubbing zone contains at least one of a scrubbing ionic liquid, water and caustic, and wherein the scrubbing zone comprises a vessel containing a tray, a distributor plate, or combinations thereof. An embodiment of the invention is one, any or all of prior embodiments in this paragraph up through the second embodiment in this paragraph further comprising recovering at least one of the ionic liquid stream from the gravity settler, the second ionic liquid stream from the coalescer, and an ionic liquid stream from the separator. An embodiment of the invention is one, any or all of prior embodiments in this paragraph up through the second embodiment in this paragraph further comprising at least one of regenerating at least a portion of the recovered ionic liquid; and recycling at least a portion of the recovered ionic liquid to a process zone. An embodiment of the invention is one, any or all of prior embodiments in this paragraph up through the second embodiment in this paragraph further comprising at least one of passing the second treated process stream to a storage facility; passing the second treated process stream to a reaction zone as a feed stream; passing the second treated process stream to a waste treatment facility; and recovering the second treated process stream as a final product.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process for removing ionic liquid from a process stream comprising:
introducing the process stream into a coalescer to form an ionic liquid stream and a first treated process stream having a level of ionic liquid less than a level of ionic liquid in the process stream; and
introducing the first treated process stream into a separator to form a second treated process stream, the second treated process stream having a level of ionic liquid less than the level of ionic liquid in the first treated process stream, the separator selected from a filtration zone comprising sand or carbon, an adsorption zone, a scrubbing zone, an electrostatic separation zone, or combinations thereof.

2. The process of claim 1 wherein the level of ionic liquid in the second treated process stream is less than about 40 ppmw.

3. The process of claim 1 wherein the level of ionic liquid in the second treated process stream is less than about 20 ppmw.

4. The process of claim 1 wherein the level of ionic liquid in the second treated process stream is less than about 5 ppmw.

5. The process of claim 1 wherein the separator is an adsorption zone, and wherein the adsorption zone contains an adsorbent comprising at least one of an oxide, a salt, an ion exchange resin, a polymer, a fiber material, activated carbon, clay, a molecular sieve, a zeolite, or combinations thereof.

6. The process of claim 5 further comprising:
desorbing the ionic liquid from the adsorbent with a desorbent or by heating.

7. The process of claim 1 wherein the separator is the scrubbing zone, and wherein the scrubbing zone contains at least one of water and caustic.

8. The process of claim 7 wherein the scrubbing zone comprises a vessel containing a tray, a distributor plate, or combinations thereof.

9. The process of claim 1 further comprising:
introducing a process feed stream into a gravity settler to form the process stream and a gravity settler ionic liquid stream before introducing the process stream into the coalescer.

10. The process of claim 1 further comprising recovering at least one of the ionic liquid stream from the coalescer, and an ionic liquid stream from the separator.

11. The process of claim 10 further comprising at least one of:
regenerating at least a portion of the recovered ionic liquid; and
recycling at least a portion of the recovered ionic liquid to a process zone.

12. The process of claim 1 further comprising at least one of:
passing the second treated process stream to a storage facility;
passing the second treated process stream to a reaction zone as a feed stream;
passing the second treated process stream to a waste treatment facility; and
recovering the second treated process stream as a final product.

13. A process for removing ionic liquid from a process stream comprising:
introducing a process feed stream into a gravity settler to form a process stream having a level of ionic liquid less than a level of ionic liquid in the process feed stream and an ionic liquid stream;
introducing the process stream into a coalescer to form a second ionic liquid stream and a first treated process stream having a level of ionic liquid less than the level of ionic liquid in the process stream;
introducing the first treated process stream into a separator to form a second treated process stream, the second treated process having a level of ionic liquid less than about 40 ppmw, the separator selected from a filtration zone comprising sand or carbon, an adsorption zone, a scrubbing zone, an electrostatic separation zone, or combinations thereof.

14. The process of claim 13 wherein the level of ionic liquid in the second treated process stream is less than about 20 ppmw.

15. The process of claim 13 wherein the level of ionic liquid in the second treated process stream is less than about 5 ppmw.

16. The process of claim 13 wherein the separator is the adsorption zone, and wherein the adsorption zone contains an adsorbent comprising at least one of an oxide, a salt, an ion exchange resin, a polymer, a fiber material, activated carbon, clay, a molecular sieve, a zeolite, or combinations thereof.

17. The process of claim 13 wherein the separator is the scrubbing zone, and wherein the scrubbing zone contains at least one of a scrubbing ionic liquid, water and caustic, and wherein the scrubbing zone comprises a vessel containing a tray, a distributor plate, or combinations thereof.

18. The process of claim 13 further comprising recovering at least one of the ionic liquid stream from the gravity settler, the second ionic liquid stream from the coalescer, and an ionic liquid stream from the separator.

19. The process of claim 18 further comprising at least one of:
regenerating at least a portion of the recovered ionic liquid; and
recycling at least a portion of the recovered ionic liquid to a process zone.

20. The process of claim 13 further comprising at least one of:
passing the second treated process stream to a storage facility;
passing the second treated process stream to a reaction zone as a feed stream; passing the second treated process stream to a waste treatment facility; and
recovering the second treated process stream as a final product.
